(19) United States
(12) Patent Application Publication
(10) Pub. No.: US 2010/0121135 A1
(43) Pub. Date: May 13, 2010

(54) SPERM CELL SEPARATION METHODS AND COMPOSITIONS

(76) Inventors: David OKSENBERG, Atherton, CA (US); Sergey Krylov, Concord (CA); Michael Musheev, North York (CA)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/817,093

(22) Filed: Aug. 24, 2007

Related U.S. Application Data
(63) Continuation-in-part of application No. PCT/US2006/006376, filed on Feb. 24, 2006.
(60) Provisional application No. 60/656,762, filed on Feb. 24, 2005.

(51) Int. Cl. A61D 19/02; C12Q 1/68; C12P 19/34; G01N 33/566; C12N 5/076

(52) U.S. Cl. 600/35; 435/6; 435/91.1; 436/501; 435/325

(57) ABSTRACT

The present invention provides an aptamer or pool of aptamers that bind(s) to a target molecule on the surface of a mammalian sperm cell and a method for producing the aptamers. The method comprises contacting a collection of different nucleic acid molecules with the target molecule under conditions favorable for binding between at least one of the nucleic acid molecules and the target molecule, to form at least one complex comprising the nucleic acid molecule bound to the target molecule, wherein each of the nucleic acid molecules comprises at least one segment of randomized nucleotide sequences. The complexes are then separated from the unbound nucleic acid molecules and unbound target molecules, and the bound nucleic acid molecule is recovered from the separated complex. The aptamers are used to separate sperm cells based upon sperm cell qualities, such as whether the cells contain X chromosomes or Y chromosomes.

### RELATED APPLICATIONS

This application is a continuation-in-part (CIP) of PCT/US2006/006376 filed on Feb. 24, 2006, which claims priority to U.S. Provisional Patent Application No. 60/656,762, filed Feb. 24, 2005, which are both incorporated herein by reference in their entirety. This application also claims priority to U.S. Provisional Patent Application No. 60/824,069, filed Aug. 30, 2006, which is herein incorporated by reference in its entirety.

### FIELD OF THE INVENTION

The present invention relates to methods and compositions for identifying and separating cells from a population of cells, particularly, mammalian sperm cells through the use of nucleic acids, particularly aptamers.

### BACKGROUND OF THE INVENTION

The ability to select sperm cells having desired characteristics remains an important objective in artificial reproduction. An efficient and cost effective process for identifying and separating sperm cells for sex selection would have significant economic implications for the livestock industry and in particular in the beef and dairy industry. For instance, in the beef industry, male bulls have greater commercial value than female cattle because of their size, so methods that allow for enrichment of male bulls would provide a clear competitive advantage to ranchers who use such techniques. On the other hand, in the dairy industry, milk-producing cows are generally more desirable. Currently however, only a small percentage of cattle ranchers employ artificial insemination methods involving sex-specific sperm cells as a means to produce livestock having the desired sex. Despite the advantages of being able to control and plan the sexual makeup of an inventory of cattle, such an approach is not more widely used in the industry because current methods for sorting sperm cells into sex-specific sperm cells, which employ flow cytometry techniques, are both expensive and involve irreversible staining of the sperm cells prior to insemination. Less costly and intrusive methods for identifying and separating sperm cells based on sperm cell quality, physical characteristics, or content would also have important applications in many animal and human reproductive technologies.

### SUMMARY OF THE INVENTION

The present invention provides at least one aptamer that binds to a target molecule accessible from the surface of a cell, particularly, a mammalian sperm cell and a method for producing the aptamers. The method comprises contacting a collection of different nucleic acid molecules with the target molecule under conditions favorable for binding between at least one of the nucleic acid molecules and the target molecule, to form at least one complex comprising the nucleic acid molecule bound to the target molecule, wherein each of the nucleic acid molecules comprises at least one segment of randomized nucleotide sequences. The complexes are then separated from the unbound nucleic acid molecules and unbound target molecules, and the bound nucleic acid molecule is recovered from the separated complex.

The present invention also provides a method for using the aptamers to identify and select sperm cells. Preferably, the method comprises separating the mammalian sperm cells by contacting the sperm cells with at least one aptamer of the invention and separating the cells into two or more populations based upon their ability to bind to the aptamer.

The invention further includes the sperm cell population produced by the separation method. The invention further comprises an artificial insemination kit comprising the sperm cell population produced by the method of the invention, and a method for artificial insemination of a mammal by administering the selected sperm cell population to the mammal.

The present invention also provides diagnostic and technological means relating to sperm cell qualities such as sperm cell viability, motility, functionality, stimulation, and preservation, as well as diagnostic and technological means that address insemination rates, fertilization rates, and birth rates of desirable offspring, by establishing a method of selectively sorting semen or sperm cells obtained from various species, individuals, and specimens.

### DETAILED DESCRIPTION OF THE FIGURES

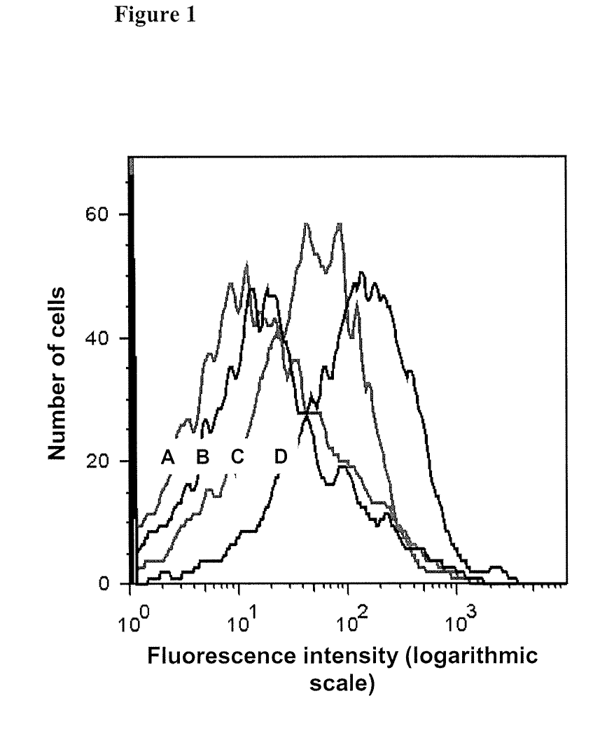

FIG. 1 is a flow cytometry graph showing the specificity of DNA aptamer pools to live sorted bull spermatozoa after 9 aptamer selection rounds (A=Y sorted bull semen cells binding to a naïve DNA library; B=Y sorted bull semen cells binding to the X cell specific aptamer pool; C=X sorted bull semen cells binding to a naïve DNA library; and D=X sorted bull semen cells binding to the X cell specific aptamer pool).

### DETAILED DESCRIPTION OF THE INVENTION

Within the context of the present invention, aptamers are defined as nucleic acid ligands; i.e., nucleic acids that bind specifically to a particular target molecule, such as a polypeptide, a short peptide, an enzyme, a protein, a lipid, a glycolipid, a phospholipid, a glycoprotein, a carbohydrate, a small molecule or a cell surface molecule, such as a receptor, an extracellular matrix or scaffolding molecule or an ion channel. The nucleic acids may be either DNA, RNA, single-stranded or double-stranded, and any chemical modifications thereof. Modifications include, but are not limited to, those that provide other chemical groups that incorporate additional charge, polarizability, hydrogen bonding, electrostatic interaction, or fluxionality to the nucleic acid bases or to the nucleic acid molecule as a whole. Preferably, the aptamers are DNA molecules. Most preferably, they are oligonucleotides. Typically, such oligonucleotides are about 15-60 nucleotides long.

Within the context of the present invention, aptamers are understood to include both monoclonal aptamers and polyclonal aptamers. As used herein, monoclonal aptamers are ones with the same nucleotide sequences that bind to the same target, and polyclonal aptamers are ones with some variation in nucleotide sequences, which bind to the same target. Monoclonal aptamers are preferred. As used herein, an aptamer, preferably is an oligonucleotide that is capable of complexing or binding to a target molecule as described herein. The affinity or complexation of an aptamer to its target molecule is a matter of degree is measured by the ability of the aptamer to bind to the target molecule at a higher degree than to a control or contaminating molecules. Thus, specificity of aptamers is similar in meaning to the specificity as it applies to antibodies.

Aptamers for sperm cell surface proteins or other sperm cell surface targets (including, but not limited to, carbohydrates, lipids, nucleotides and other small molecules that may be accessible to bind to specific aptamers) are identified and selected by the methods of the invention. In one embodiment, the invention provides a method for producing an aptamer that binds to a target molecule on the surface of a mammalian sperm cell. A collection of different nucleic molecules is contacted with a target molecule that is accessible for binding to these molecules, likely on the cell surface or protruding from the cell surface, under conditions favorable for binding between at least one of the nucleic acid molecules and the target molecule. Each of the nucleic acid molecules contains at least one segment of randomized nucleotide sequences. This provides variation in the nucleic acid molecules. The contact results in the formation of at least one complex comprising at least one nucleic acid molecule bound to the target molecule. The complexes are then separated from the unbound nucleic acid molecules and the unbound target molecules. Then, the bound nucleic acid molecule is recovered from the separated complex, thus providing the desired aptamer. In a further embodiment, the method comprises the further step of amplifying the recovered nucleic acid molecules to create additional molecules. In a further, preferred embodiment, the recovered and amplified molecules, i.e., aptamers, are further mixed with the collection of target molecules accessible for binding, preferably on or accessible from the cell surface and the sequence of steps stated above is repeated a sufficient number of times until aptamers of a desired specificity and binding affinity are recovered. The aptamers bind more specifically with a target molecule on a cell as compared to a control pool of DNA sequences. In an alternative and preferred embodiment, the contacting step comprises incubating the molecules to form an equilibrium mixture and the separating step comprises capillary electrophoresis.

Preferably, the target molecule on the surface of a cell is one that is unique to either a sperm cell containing a Y chromosome or a sperm cell containing an X chromosome and that will not cross react with each type of cell. Such a target molecule, can be a protein, a peptide, an enzyme, a lipid, a glycolipid, a phospholipid, a glycoprotein, a carbohydrate, a small molecule or a cell surface molecule, such as a receptor, an extracellular matrix or scaffolding molecule or an ion channel. Most preferably, the protein distinguishes sperm cells containing a Y chromosome from sperm cells containing an X chromosome.

In one embodiment of the invention, the methods of the invention are performed in an iterative fashion such that there are repeated steps of (a) contacting a collection of different nucleic acids (i.e., aptamers) with a target molecule on the surface of a cell to form at least one complex comprising at least one nucleic acid molecule bound to a target molecule on the surface of a cell, (b) separating the complexes from unbound nucleic acid molecules and unbound target molecules; and (c) recovering the bound nucleic acid molecule from the separated complex. In this embodiment, a plurality of aptamers recovered from the separated complex of step (c) are used in a subsequent round of the method in step (a). In one embodiment of the invention, the steps of the sorting sperm cells (i.e., steps (a), (b) and (c) above) are repeated 1 or more, 2 or more, 3 or more, 4 or more, 5 or more, 6 or more, 7 or more, 8 or more, 9 or more, 10 or more, or 11 or more times to produce an aptamer pool specific for Y containing or X containing sperm. Sorted aptamers can be purified by methods known in the art.

The present method further comprises the step of amplifying the recovered nucleic acid molecule to create additional nucleic acid molecules. The contacting step (a) comprises incubating the molecules to form an equilibrium mixture and wherein the separating step comprises capillary electrophoresis.

The present method further comprises incubating the collection of different nucleic acid molecules or aptamers after each round and the target molecule with a primer that is substantially complementary to template nucleic acid sequence. The template nucleic acid sequence is a sequence that corresponds to a segment of said aptamer that is unvaried within a plurality of aptamers. The aptamer preferably is from a DNA library and this template nucleic acid sequence is a vector sequence. The contacting step (a) further comprises a negative selection step followed by a positive selection step. The plurality of the recovered aptamers are quantified using flow cytometry.

The target molecule to which the aptamer can bind is selected from the group consisting of a protein, a peptide, an enzyme, a lipid, a glycolipid, a phospholipid, a glycoprotein, a carbohydrate, a small molecule or a cell surface molecule. In a preferred embodiment the target molecule is a target molecule contained on a sperm cell and the target molecule distinguishes at least one sperm cell containing a X chromosome from at least one sperm cell containing an Y chromosome. The sperm cells identified by this method are cattle sperm cells or human sperm cells.

Aptamers that specifically bind to sperms cells can be identified by methods known in the art, including, but not limited to cell sorting and flow cytometry techniques. The present method provides a method of preparing aptamers permit separation of Y chromosome-bearing mammalian sperm cells from X chromosome-bearing mammalian sperm cells, wherein the method produces a first group of aptamers that bind to Y chromosome-bearing sperm cells in the first sample and a second group of aptamers that bind to X chromosome-bearing mammalian sperm cells in the second sample; and the method further comprises: (d) comparing the first and second groups of aptamers to identify by process elimination at least one aptamer that binds only to either the Y chromosome-bearing mammalian sperm cells or the X chromosome-bearing mammalian sperm cells. The first and second samples of mammalian sperm cells are produced by flow cytometry and cell sorting.

The method of preparing sperm specific aptamers further comprises a contacting step (a) which further comprises: (a1) contacting a first collection of different nucleic acid molecules with a target molecule contained in a first sample of Y chromosome-bearing mammalian sperm cells under conditions favorable for binding between the nucleic acid molecules and the Y chromosome-bearing sperm cells to form at least one complex comprising at least one nucleic acid molecule bound to at least one Y chromosome-bearing sperm cell, wherein each of the nucleic acid molecules comprises at least one segment of randomized nucleotide sequences; and (a2) contacting a second collection of different nucleic acid molecules with a target molecule contained in a second sample of X chromosome-bearing mammalian sperm cells under conditions favorable for binding between said nucleic acid molecules and said X chromosome-bearing sperm cells to form at least one complex comprising at least one nucleic acid molecules bound to at least one X chromosome-bearing sperm cell, wherein each of said nucleic acid molecules comprises at least one segment of randomized nucleotide sequences; wherein the separation step (b) and the recovery step (c) thereby produces aptamers for Y chromosome-bearing mammalian sperm cells and aptamers for X chromosome-bearing mammalian sperm cells. The aptamers recovered by this method are those that bind to Y chromosome-bearing mammalian sperm and those that bind to X chromosome-bearing mammalian sperm.

The method of producing sperm specific aptamers further comprises that prior to step (a), at least 1 round of contact of the nucleic acid molecules with an unsorted cell population comprising both X-chromosome-bearing mammalian cells and Y-chromosome-bearing mammalian cells occurs. Also the separating step (b) further comprises the step of separating said sperm cells bound to the aptamer from the aptamer and the recovering step (c) further comprises recovering the separated sperm cells. The mammalian sperm cells preferably are cattle sperm cells or human sperm cells.

In one embodiment of the invention, rounds of unsorted aptamer selection can be performed prior to rounds of sorted aptamer selection. For instance, 1 to 5 rounds of unsorted aptamer selection can be performed. The invention includes methods comprising 3 rounds of unsorted aptamer selection prior to rounds of sorted aptamer selection. The method of claim 1, further comprises incubating the collection of different nucleic acid molecules and the target molecule with a primer that is substantially complementary to template nucleic acid sequence.

In an alternative and preferred embodiment, the invention provides a method for producing an aptamer that permits separation of Y chromosome-bearing mammalian sperm cells from X chromosome-bearing mammalian sperm cells. A first sample of Y chromosome-bearing mammalian sperm cells is obtained, and a second sample of X chromosome-bearing mammalian sperm cells is obtained. A first group of aptamers are produced, which bind to the Y chromosome-bearing mammalian sperm cells in the first sample, and a second group of aptamers are produced that bind to the X chromosome-bearing mammalian sperm cells in the second sample. The first and second groups of aptamers are compared to identify by process of elimination at least one aptamer that binds to either of the Y chromosome-bearing mammalian sperm cells or the X chromosome-bearing mammalian sperm cells. Generally, several different aptamers are identified that bind to either type of cells.

Preferably, the first and second samples of mammalian sperm cells, which contain either Y chromosome-bearing sperm cells or X chromosome-bearing mammalian sperm cells are produced by flow cytometry and cell sorting techniques that are known to those skilled in the art. Such techniques are disclosed in U.S. Pat. No. 5,135,759, issued Aug. 4, 1992. Generally, a flow cytometer measures the amount of fluorescent light given off when the sperm, previously treated with a fluorescent dye, passes through a laser beam. The dye binds to the DNA. Because the X chromosome contains more DNA than the Y chromosome, the female (X) sperm takes up more dye and gives off more fluorescent light than the male (Y) sperm. To detect the small differences in DNA between the X and the Y sperm, the sperm passes single file through the laser beam, which measures the DNA content of individual sperm. This permits separation of the individual X and Y chromosome-containing sperm by a cell sorter. Sorted X chromosome-bearing and Y chromosome-bearing sperm cells can be purchased from commercial sources which have been sorted using similar cell sorting methods.

Preferably, the aptamers are produced by a process comprising the steps of: (a) contacting a first collection of different nucleic acid molecules with a first sample of Y chromosome-bearing mammalian sperm cells under conditions favorable for binding between the nucleic acid molecules and the Y chromosome-bearing sperm cells to form at least one complex comprising at least one nucleic acid molecules bound to at least one Y chromosome-bearing sperm cell, wherein each of the nucleic acid molecules comprises at least one segment of randomized nucleotide sequences; (b) contacting a second collection of different nucleic acid molecules with the second sample of X chromosome-bearing mammalian sperm cells under conditions favorable for binding between the nucleic acid molecules and the X chromosome-bearing sperm cells to form at least one complex comprising at least one nucleic acid molecule bound to at least one X chromosome-bearing sperm cell, wherein each of the nucleic acid molecules comprises at least one segment of randomized nucleotide sequences; (c) separating the complexes from the unbound nucleic acid molecules and unbound target molecules; and (d) recovering the bound nucleic acid molecules from the complexes, thereby producing aptamers for Y chromosome-bearing mammalian sperm cells and aptamers for X chromosome-bearing mammalian sperm cells.

In a preferred embodiment, the aptamers are tested and validated by contacting them with a sample of sperm cells containing sperm cells that have X chromosomes and sperm cells that contain Y chromosomes, separating the sperm cells by flow cytometry and cell sorting, and determining that the putative X-binding aptamer binds to the X sperm cell and the putative Y-binding aptamer binds to the Y sperm cell. Generally, the aptamers are labeled, for example, with a fluorescent moiety to permit the appropriate identification.

In producing the aptamers of the invention various specific techniques known to those skilled in the art may be used. One such technique is the MonoLex process of AptaRes, Luckenwalde, Germany. The process involves the steps of: (1) synthesis of an oligonucleotide library with regions of random sequence; (2) affinity adsorption of the oligonucleotides to a target; (3) affinity sorting of the oligonucleotides along an affinity resin; (4) separation of the oligonucleotides with different levels of affinity into numerous pools comprising multiple aptamers per pool; (5) amplification of the separated nucleotide pools (which produces polyclonal aptamers); and (6) identification of individual oligonucleotides by cloning and sequencing (which produces monoclonal aptamers).

Another technique is known as the SELEX (Systematic Evolution of Ligands by EXponential enrichment) process. The SELEX process and variants thereof are described in U.S. Pat. Nos. 5,861,254; 6,261,774 B1; 6,376,190 B1; 6,506,887 B1; 6,706,482 B2 and 6,730,482 B2. This process includes the steps of: (1) contacting a mixture of nucleic acids, preferably comprising segments of randomized sequences, with a target under conditions favorable for binding; (2) partitioning unbound nucleic acids from those nucleic acids that have bound specifically to target molecules; (3) disassociating the nucleic acid-target complexes; (4) amplifying the nucleic acids disassociated from the nucleic acid-target complexes to yield a ligand-enriched mixture of nucleic acids; and (5) repeating the previous steps through as many cycles as desired or necessary to yield highly specific, high affinity aptamers to the target molecule.

A related process is the CE-SELEX (capillary electrophoresis-SELEX) process as described in J. Am. Chem. Soc. 2004, 126, 20-21. This technique uses electrophoresis to separate binding sequences from inactive ones. Selection occurs in free solution. Active sequences that bind the target undergo a mobility shift, similar to that seen in affinity capillary electrophoresis. Active sequences are separated from inactive sequences and collected as separate fractions.

A preferred method for identifying and isolating aptamers is the NECEEM (NonEquilibrium Capillary Electrophoresis of Equilibrium Mixtures) process as described in J. Am. Chem. Soc. 2002, 124, 13674-13675, Anal. Chem. 2003, 75, 1382-1386, Krylov, “NECEEM for Development, Characterization and Analytical Utilization of Aptamers,” Lab Plus International, November 2005, and Krylov, “Nonequilibrium Capillary Electrophoresis of Equilibrium Mixtures (NECEEM): A Novel Method for Biological Screening,” J. Biomol. Screen Online First, Jan. 17, 2006.

Briefly, this method starts with a naive DNA library (every sequence is statistically unique) that is mixed with the target protein and incubated to form an equilibrium mixture. DNA molecules with high affinity bind to the target protein, while those with low affinity do not bind. A plug of the equilibrium mixture is then introduced into a capillary, and a high voltage is applied. The equilibrium fraction of DNA-target is separated from the equilibrium fraction of DNA by gel-free capillary electrophoresis under non-equilibrium conditions. Under these conditions, the mobility of the target is higher than that of DNA, and the mobility of the target-DNA complex is typically intermediate between that of the DNA and the target. In the electric field, the zones are thus separated, and equilibrium between the three components is no longer maintained. The DNA-target complex starts disassociating, which results in “smears” of DNA and target between three peaks. Due to the high efficiency of separation, reattachment of disassociated DNA and target is negligible.

The components reach the end of the capillary in the following order: (1) the equilibrium part of free target; (2) free target formed by disassociation of DNA-target during NECEEM; (3) the remains of intact DNA-target; (4) free DNA formed from the disassociation of DNA-target during NECEEM; and (5) the equilibrium part of free DNA. A fraction is collected from the output of the capillary in a time window. The widest aptamer collection window includes DNA-target complexes and DNA disassociated from DNA-target complexes during NECEEM.

The preferred targets are polypeptides (peptides and proteins) on the sperm that are sex-specific or otherwise allow identification or separation of the sperm based on sex selection or other desired characteristics. Examples of such polypeptides are disclosed in U.S. Pat. Nos. 4,191,749; 4,448,767; 5,021,244; 6,153,373; and 6,489,092 and in U.S. Patent Application Publication 2003/0162238 A1. For example, the published patent application discloses an isolated sex-chromosome-specific protein characterized as follows: (a) X chromosome specific, (b) associated with the cell membrane of bovine sperm cells, and (c) having a molecular weight on SDS-PAGE of about 32 kDa.

Once suitable aptamers are selected, they may be produced and reproduced by many techniques well known to those of ordinary skill in the art, including enzymatic techniques or through chemical synthesis. Additional chemical groups may be added through known chemical techniques. Such groups include fluorescent and other groups that create a detectable signal. In addition, modified nucleotides may be used to protect the aptamers from degradation by nucleases. Such modified nucleotides include 2′-0-methyl and 2″-fluro derivatives.

In some embodiments, the aptamers selected and/or produced as described above will be used in an assay for separating populations of sperm cells based on whether they carry the X or Y chromosome, an abnormal number of sex chromosomes, or other desired sperm cell characteristics. In a preferred embodiment, the method of separating mammalian sperm cells comprises the steps of: 1) contacting the mammalian sperm cells with the aptamer of the invention and 2) separating the sperm cells into two or more populations based on the ability of the sperm cells to bind to the aptamer. In one embodiment, the method comprises the further step of separating the sperm cells bound to the aptamer from the aptamer and recovering the separated sperm cells. In another embodiment, the sperm cells that are not bound to the aptamer are recovered.

In an alternative embodiment, more than one type of aptamer maybe used. Thus, two or more aptamers of the invention may be used in the separation method. Aptamers produced by the present method are useful for binding to target molecules and more specifically those target molecules on sperm cells, both X containing sperm cells and Y containing sperm cells.

This method produces sperm cell populations selected and separated based upon certain desired characteristics of the cells. Separated populations maybe bound or unbound to the aptamer. If the desired population is bound to the aptamer, preferably the sperm cells are separated from the aptamer molecules. Preferably, the sperm cells are cattle sperm cells. In one preferred embodiment, the sperm cells contain a Y chromosome. In an alternative preferred embodiment, the sperm cells contain only the X chromosome.

An assay for separating sperm cells by use of aptamers could be used, for example, as follows. Commercially available, microscopically small magnetic beads are coated with an appropriate aptamer of the invention, such as X chromosome-bearing sperm specific aptamers. These beads will be placed in a suspension of the sperm-cells in an appropriate receptacle, such as a glass dish. Because the sex-chromosome-specific proteins are present on the cell surface, the X chromosome bearing sperm cells will then bind to the female specific aptamer on the beads, while the Y chromosome bearing sperm cells will not. The beads are then pulled to the side of the dish using a magnet. Sperm cells having the Y chromosome are then recovered. Other types of beads that can be used to capture and recover the aptamers may also be used. Additional techniques using magnetic beads coated with substances that bind to sperm cells are disclosed in U.S. Patent Application Publication Nos. 2003/0068654 A1, 2004/0142384 A1, and 2005/0114915 A1.

In another example, agglutination of sperm cells may be used. In such an approach, live, unsorted sperm may be suspended in a serum free, in vitro culture medium and exposed to either Y or X chromosome sperm cell specific aptamers. Following treatment, the medium is filtered in a glass wool filter, and sperm in the filtrate is used to perform in vitro fertilization.

In another example, the aptamers against X or Y chromosome sperm cell specific surface proteins may bind to and inactivate X or Y sperm cells respectively, and may prevent them from fertilizing an ovum. The sperm cells not bound by the aptamers may remain viable and active for fertilizing ova. Thus, the invention provides a method to produce a semen sample enriched in active X or Y sperm cells and thus capable of increasing the probability that offspring will be of a desired sex or have a gene for a sex-chromosome linked trait.

In another example, a native sperm preparation may be exposed to a first aptamer that binds, for example, male specific molecules. The exposed sperm may be suspended together with a conjugate of a second aptamer that binds exclusively to the first aptamer and an immunoabsorbent substrate in a protein-free diluent to form a conjugate/sperm preparation whereby the male sperm are bound to the substrate. The male sperm may then be recovered from the substrate by specific binding of the substrate.

The methods described herein provide the means to separate sperm on factors of quality and desirability, including sperm cell motility, functionality, stimulation, and preservation, which can affect fertility rates, insemination rates, fertilization rates, offspring health, and offspring desirability for various species of mammals, including, but not limited to, humans, horse, cattle, swine, cats, dogs, buffalo, oxen, and elk.

The methods for separating sperm on the basis of desired characteristics described herein minimize damage to the sperm by mechanical handling so that the sperm have improved viability. The methods are non-invasive, do not require chemical binding to cellular internal structures, involve minimal manipulation, and are inexpensive. There are minimal requirements for equipment or instrumentation and they are readily carried out by a person skilled in the art.

The aptamers prepared by the present method and methods of the present invention may also be used to evaluate other characteristics of the sperm. For example, they may be used to determine sperm quality, determine male fertility, identify healthy sperm, or identify abnormal or damaged sperm.

The separated sperm cells of the invention are preferably used for artificial insemination of a mammal. Preferably, the mammal is a bovine mammal. The method for artificial insemination comprises administering the sperm to the mammal using techniques known to those skilled in the art.

The invention further comprises a kit for artificial insemination of a mammal. The kit contains at least the separated sperm cell population of the invention and optionally other components or devices to administer the sperm cell population to the mammal. Preferably, the kit contains the individual sperm cell sample in a tube for insertion into the vagina of the female animal. Such sample tube is known in the art as a “straw”.

Alternatively, the separated sperm of the invention maybe used for in vitro fertilization of a mammal. Preferably, the mammal is a human being.

### EXAMPLES

### Sperm Aptamers Selection Method

A preferred method of selecting specific aptamers according to the present invention employs a method that utilizes several rounds of incubation of unsorted, sorted X and/or sorted Y sperm cells with a DNA library with a final step of collecting the solution with DNA binders and use it as a template for PCR and for strand separation on streptavidin beads. Applicants have determined that more than 6 rounds of sperm aptamer selection according to the present invention is optimal. Preferably 7 rounds, more preferably 8 rounds and even more preferably 9 rounds of selection are performed as outlined below. However, more than 9 rounds of selection can be performed if necessary to obtain aptamers that bind to the specific target molecule. It should be understood that the protocol described below can vary in temperature, volume, and other parameters that do not effect the outcome of the protocol but that will result in the production of specific aptamers that bind to target molecules. Such variations are known to persons skilled in the art.

### Rounds 1-3 of Aptamer Selection

### Preparation of Cells:

Thaw the sperm aliquot by placing the straw in 37° C. water for 1 min, and then keep the cells at room temperature (20-25° C.)(RT).
1. Take 1 straw of unsorted sperm cells (about 0.5 ml and 10⁷ cells).
2. Add 1 ml of the PVA (polyvinyl alcohol) containing semen buffer.
3. Spin the sperm cells for 12 min at 300×g at 17° C. in a 1.5 ml vial.
4. Remove the buffer and re-suspend cells in 1 ml of fresh PVA-semen buffer.
5. Spin the sperm cells for 12 min at 300×g at 17° C. in a 1.5 ml vials.
6. Resuspend the sperm cells in 300 μl, of the fresh PVA-semen buffer and count the cells using chemocytometer. Dilute the cells to ˜2×10⁶ cells/ml (40 000 cells per 20 μL) by adding 600 μL of buffer.
7. Anneal 50 μM of naive DNA library (custom ordered from Integrated DNA Technologies (IDT), Coralville, Iowa) with the following sequence: 5′-CTC CTC TGA CTG TAA CCA CG -(40N)- GGC TTC TGG ACT ACC TAT GC -3′ dissolved in PBS (phosphate buffered saline buffer) with 2.5 mM MgCl₂ by heating to 94° C. for ˜3 min, then cooling to RT.
8. Add 2 μL of 1 mM F-primer (forward primer custom ordered from IDT with the following sequence 5′-CTC CTC TGA CTG TAA CCA CG -3′) into the 40 μL aliquots of cells, to a final concentration of 50 μM. Fprimer is a first 20 nucleotides of the library. It is used in the selection as a background DNA. This sequence was chosen to decrease the likelihood that at least one of the constant regions of the library will not participate in the binding, and if removed will produce a shorter aptamer sequence but will not change the binding properties.
9. Add 4 μL of 100 μM annealed library into the 20 μL aliquot containing sperm cells with 50 μM F-primer.
10. Incubate for 1 hour at room temperature.
11. Add 200 μL of fresh PVA-semen buffer and spin for 12 min at 300×g at 17° C.
12. Remove the supernatant and re-dissolve the pellet in additional 200 μL of the PVA-semen buffer.
13. Spin for 12 min at 300×g at 17° C.
14. Remove the supernatant and re-dissolve the pellet in additional 200 μL of the PVA-semen buffer.
15. Spin for 12 min at 300×g at 17° C.
16. Remove the supernatant and add 20 μL of the 10 mM Tris-HCl buffer, pH 7.5 and incubate cells at 95° C. for 5 min.
17. Spin down the cellular debris for 20 min at 20 000 rpm.
18. Collect the supernatant and use it as template for PCR using F primer and biotinylated R-primer (reverse primer custom ordered from IDT with the following sequence: 5′-biotin-GGC TTC TGG ACT ACC TAT GC -3′) and perform strand separation on streptavidin magnetic beads.

Completion of steps 1-18 above constitutes the completion of a single round. The supernatant from 18 is considered to be the Round 1 (R1) aptamer pool and is used directly or is amplified using known PCR techniques. For Round 2 (R2), the R1 aptamer pool is used in place of the naive DNA library in step 7 on a new sample of unsorted cells that are prepared according to steps 1-6. The supernatant or Round 2 (R2) aptamer pool from step 18 of the completion of Round 2 is then utilized directly or amplified using known PCR techniques and added in place of the DNA library in step 7 on a new sample of unsorted cells in Round 3 (R3). The supernatant from step 18 of Round 3 or the Round 3 (R3) aptamer pool is then used in step 7 of Round 4 described below.

Rounds 4-11 (Positive and Negative Selections with Sorted Cells)

### Preparation of Cells:

Thaw the sperm aliquots (sorted X or sorted Y containing cells) by placing the straw in 37° C. water for 1 min, and then keep the cells at room temperature (20-25° C.),
1. Take 1 straw of sorted X and 1 straw of sorted Y cells,
2. To each tube, then add 200 μl of fresh PVA semen buffer, and spin for 12 min at 300×g at 17° C., remove the supernatant leaving about 10 μl add 200 μl of fresh PVA-semen buffer and transfer all into the 200 μl PCR vial.
3. Spin the cells for 12 min at 300×g at 17° C. in a 0.2 ml PCR vials (smaller vials improves cell recovery) and remove the buffer leaving about 20 μl.
4. To each tube, add 60 μl of fresh PVA Semen buffer and split in 2 tubes by 30 μl.
5. Count the cells using chemocytometer. It should be around 2×10⁶ cells/ml (4×10⁴ cells per 20 μl).
6. About 1-2 min before addition of the R3 Aptamer pool (see step 18 of Round 3 above) to the cell suspension, add the F-primer to the final concentration of 50 μM (add 1 μl of 1 mM to 20 μL of cells).
7. For negative selection make the following mixtures:
   A. Y cells with 50 μM of F4 and 5 nM of X Apt pool from previous selection round (or R3 aptamer pool only if it is forth selection round).
   B. X cells with 50 μM of F4 and 5 nM of Y Apt pool from previous selection round (or R3 aptamer pool only if it is forth selection round). Incubate the mixtures at room temperature for 1 hour and spin the cells down (300×g, 17° C. 12 min). Take the supernatant and use it in the positive selection. To each one of X and Y cells aliquots, add F-primer for a final concentration of 50 μM and equal amount of the supernatant from previous step.
   C. To X cells, add supernatant from fraction 1 to get 2.5 nM X aptamer pool and 50 μM F4.
   D. To Y cells, add supernatant from fraction 2 to get 2.5 nM Y aptamer pool and 50 μM F4.
8. Incubate the mixtures at room temperature for 1 hour.
9. Add 200 μL of fresh PVA-semen buffer and spin for 12 min at 300×g at 17° C.
10. Remove the supernatant and re-dissolve the pellet in additional 200 μL of the PVA-semen buffer.
11. Spin for 12 min at 300×g at 17° C.
12. Remove the supernatant and re-dissolve the pellet in additional 200 of the PVA-semen buffer.
13. Spin for 12 min at 300×g at 17° C.,
14. Remove the supernatant and add 20 μL of the buffer and incubate cells at 95° C. for 5 min.
15. Spin down the cellular debris for 20 min at 20 000 rpm.
16. Collect the supernatant or aptamer pool and use it as template for PCR and strand separation on streptavidin magnetic beads.

A round of the aptamer selection of Rounds 4-9 and higher rounds include steps 1-16 directly above. The Round 4 aptamer pool is then added to step 7 of the next round, Round 5, then the Round 5 aptamer pool is then added to step 7 of the next round, Round 6, then the Round 6 aptamer pool is then added to step 7 of the next round, Round 7, then the Round 7 aptamer pool is then added to step 7 of the next round, Round 8, then the Round 8 aptamer pool is then added to step 7 of the next round, Round 9, then the Round 9 aptamer pool is then added to step 7 of the next round, and any additional rounds can continue to be repeated accordingly.

The goal of these rounds of aptamer selection is to obtain a pool of aptamers that bind specifically to Y containing sperm or X containing sperm. Therefore, at least Round 7 of the selection method should be run to obtain aptamers that specifically bind to the target molecule, more preferably at least Round 8 should be run and most preferably at least Round 9 or higher should be run to obtain a pool of aptamers that are specific for Y containing and X containing sperm.

Applicants submit that the above method can be utilized to prepare target specific aptamers of any type as long as the pool of cells that contain the target molecule that identifies the cell (unsorted or sorted) is substituted in the rounds of aptamer selection for the Y and X containing sperm both unsorted and sorted.

### Cell Cytometry Analysis of Aptamers Produced in Aptamer Selection Method

The following method was performed to provide labeled pools of DNA and to provide a sufficient amount of DNA for analysis that could additionally save time and costs in the preparation. The known technique, asymmetric PCR, resulted in the production of one of the DNA strands. In order to determine if a pool of aptamers were good binders, labeled aptamers resulting from the asymmetric PCR technique were mixed with sperm cells and subjected to flow cell cytometry analysis. Naïve DNA library was used as a control.

Prepare the following mixture containing: 1 μM Alexa 647 (obtained from IDT as a custom primer) labelled forward primer (5′-Alexa 647-CTC CTC TGA CTG TAA CCA CG -3′), 50 nM reverse primer (5′-GGC TTC TGG ACT ACC TAT GC -3′) 50 mM KCl, 10 mM Tris-HCl (pH 8.6), 2.5 mM MgCl2, 200 μM of each deoxyribonucleotide triphosphate (dNTP), and 0.05 unit/μL Taq DNA polymerase.

There were 20 PCR cycles performed which includes a PCR cycle consisting of melting at 94° C. for 10 seconds, annealing at 56° C. for 10 seconds, and extension at 72° C. for 10 seconds. The first cycle has an extended melting step of 30 seconds. As a template, the purified single stranded aptamers pools after each selection round are used. After PCR is completed, the products are purified using the 30 kDA cut off DNA purification column (Microcon ultracel YM-30, from Millipore Bedford Mass., USA.) using standard procedure described in the manual and known to persons skilled in the art.

Preparation of cells: Thaw the sperm aliquot by placing the straw in 37° C. water for 1 min, and then keep the cells at room temperature (20-25° C.).

Take 1 or more straws of unsorted cells (about 0.5 ml and 10⁷ cells)
1. Add 1 ml of the PVA semen buffer.
2. Spin the cells for 12 min at 300×g at 17° C. in a 1.5 ml vial.
3. Remove the buffer and re-suspend cells in 1 ml of fresh PVA buffer.
4. Spin the cells for 12 min at 300×g at 17° C. in a 1.5 ml vial.
5. Re-suspend cells in 300 μl of the fresh PVA containing semen buffer and count the cells using haemocytometer.
6. Dilute cells to ˜2×10⁶ cells/ml (20 000-40 000 cells per 20 μL); and
7. 200 μl of cells+10 μl of 1 mM F4 primer then aliquot by 20 μl each.

In parallel:
1. Take 1 or more straws of sorted X and 1 or more straws of sorted Y cells.
2. Spin for 12 min at 300×g at 17° C., remove the supernatant leaving about 10 μL add 200 μl of fresh PVA Semen buffer and transfer all into the 200 μl PCR vial.
3. Spin the cells for 12 min at 300×g at 17° C. in a 0.2 ml PCR vials (smaller vials improves cell recovery) and remove the buffer leaving about 20 μl.
4. To each tube, then add 200 μl of fresh PVA Semen buffer.
5. Count the cells using haemocytometer it should be around 2×10⁶ cells/ml (4×10⁴ cells per 20 μL).
6. Mix X cell mix with 1 mM F primer for a final concentration of 50 μM and make as many 20 μL aliquots as required for analysis.
7. Mix Y cell mix with 1 mM F primer for a final concentration of 50 μM and make as many 20 μL aliquots as required for analysis.
8. Mix unsorted cells with 1 mM F primer for a final concentration of 50 μM and make as many 20 μL aliquots as required for analysis.
9. Anneal the purified DNA libraries, and pools dissolved in PBS with 2.5 mM MgCl₂. (heat to 94° C. for ˜3 min, then cool down at RT).
10. Add appropriate amounts of each aptamer pool from the rounds of aptamer selection described above that is to be tested into the 20 μL aliquot containing sperm cells with 50 μM F4 to a final concentration of ˜10 nM: Make the following mixtures: unsorted, sorted X and sorted Y cells each with Naive DNA library and the pools from each selection round.
11. Incubate for 1 hour at room temperature.
12. Add 200 μL of fresh PVA-semen buffer and spin for 12 min at 300×g at 17° C.
13. Remove supernatant and add 200 μL of fresh PVA-buffer and transfer everything to the cell cytometry tube, and
14. Add the PI (propidium iodide, a nucleic acid stain and a dead cell marker, Sigma Aldrich) about 2-3 min before the analysis and additional 200 μL of the PVA-semen buffer.

FIG. 1 provides the analysis of the aptamers obtained by the present method in which 9 rounds of aptamer selection and analysis using flow cytometry was performed. Each population of live spermatozoa was represented by 8,000 to 9,000 cells. Live cells population were differentiated from dead cells by Propidium Iodide, a dead cell stain.

Comparing the binding of X sorted cells to the naïve library and to the X aptamer pool (FIG. 1, traces C and D, respectively), the results show that the X aptamer pool has about 10 times greater affinity to the X cells then the naïve library. This data shows that sperm cell specific aptamers were obtained from the library during the selection process. Then, the comparison of the binding affinities of X aptamer pool to X and Y sorted cells (FIG. 1, traces D and B, respectively) indicates that aptamer affinity to X cells is about 20 times greater then to Y cells. All the above data, together with the fact that the affinity of Y cells to the naïve DNA library, and to the X aptamer pool (FIG. 1, traces A and B, respectively) is relatively similar proves that the present method produces X cell specific aptamers, which can specifically bind to the X cells and has no, or very insignificant affinity to Y cells.

With regard to the binding of Y cells to the naïve DNA library and to the Y aptamer pool, data (not shown) shows that Y cells bind better to the Y aptamer pool than they do to the naïve DNA library which is indicative of Y specific aptamers.

Generally, the present method provides aptamers that bind to target molecules that may or may not be on the surface of a cell but should be accessible from the surface of a cell, such as preferably a X- or Y-containing sperm cells. The cell cytometry analysis described herein provides a method to determine whether an aptamer pool obtained in the last step of a single round aptamer selection round as described herein contains one or more aptamers that bind to a target molecule. One should review the data and compare the binding of the aptamer pool with a sample containing the target molecule. This binding data should be compared with any binding of the same aptamer pool to the naïve DNA library, preferably the library from which the aptamer pools were first obtained as a negative control. Additionally, the aptamer pool binding also should be compared to a second or third sample that does not contain the target molecule. Results that show higher binding of the aptamer pool to the target molecule as compared to the binding to the naïve DNA library is considered as a positive binding. Additionally, further rounds of selection as disclosed herein may increase the binding affinity of the aptamer pool. Additionally, if one wishes to separate to cell populations based on specific binding to one cell and not the other, such as with the X- and Y-containing sperm cells, then in addition to comparing the binding of the aptamer pool to the naïve DNA library, the aptamer pool binding also should be analyzed for its binding to the preferred target molecule as well as a sample containing the “non-target” molecule from which one wishes to separate the target molecule. In this latter instance, all of these comparisons should be analyzed to select aptamer pools that bind to a specific target molecules and do not bind or binds to a lesser degree to the cell or molecule from which one wants to separate the target molecule. Thus, using aptamers to separate different cell populations requires analysis and comparison to different controls.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. The use of the words “a” or “an” herein to describe any aspect of the present invention is to be interpreted as indicating one or more.

Although this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method for producing at least one aptamer that binds to a target molecule on the surface of a mammalian sperm cell comprising the steps of:
(a) contacting a collection of different nucleic acid molecules with said target molecule under conditions favorable for binding between at least one of said nucleic acid molecules and said target molecule, to form at least one complex comprising said at least one nucleic acid molecule bound to a target molecule, wherein each of said nucleic acid molecules comprises at least one segment of randomized nucleotide sequences;
(b) separating said complexes from the unbound nucleic acid molecules and unbound target molecules; and
(c) recovering the bound nucleic acid molecules from the separated complex, wherein said bound nucleic acid molecules comprise at least one aptamer that binds to said target molecule.

2. The method of claim 1 further comprising the step of amplifying the recovered nucleic acid molecule to create additional nucleic acid molecules.

3. The method of claim 1, wherein said contacting step comprises incubating said molecules to form an equilibrium mixture and wherein said separating step comprises capillary electrophoresis.

4. The method of claim 1, wherein said target molecule is selected from the group consisting of a protein, a peptide, an enzyme, a lipid, a glycolipid, a phospholipid, a glycoprotein, a carbohydrate, a small molecule or a cell surface molecule.

5. The method of claim 4, wherein said target molecule is a target molecule contained on a sperm cell.

6. The method of claim 5, wherein said target molecule distinguishes at least one sperm cell containing a X chromosome from at least one sperm cell containing an Y chromosome.

7. The method of claim 5, wherein said sperm cells are cattle sperm cells.

8. The method of claim 5, wherein said sperm cells are human sperm cells.

9. The method of claim 1, wherein said recovered aptamers of step (c) are subsequently used as the collection of different nucleic acid in step (a).

10. The method of claim 1, wherein steps (a), (b) and (c) are repeated 1 or more, 2 or more, 3 or more, 4 or more, 5 or more, 6 or more, 7 or more, or 8 or more times to produce an aptamer pool specific for Y containing or X containing sperm.

11. The method of claim 1, further comprising incubating said collection of different nucleic acid molecules and said target molecule with a primer that is substantially complementary to template nucleic acid sequence.

12. The method of claim 11, wherein said template nucleic acid sequence is a sequence that corresponds to a segment of said aptamer that is unvaried within a plurality of aptamers.

13. The method of claim 12, wherein said aptamer is from a DNA library and said template nucleic acid sequence is a vector sequence.

14. The method of claim 1, wherein step (a) further comprises a negative selection step followed by a positive selection step.

15. The method of claim 1, wherein said recovered aptamer is purified.

16. The method of claim 1, wherein a plurality of the recovered aptamers are quantified using flow cytometry.

17. The method of claim 6, wherein said aptamer permits separation of Y chromosome-bearing mammalian sperm cells from X chromosome-bearing mammalian sperm cells, wherein said method produces a first group of aptamers that bind to Y chromosome-bearing sperm cells in said first sample and a second group of aptamers that bind to X chromosome-bearing mammalian sperm cells in said second sample; and said method further comprises:
(d) comparing said first and second groups of aptamers to identify by process elimination at least one aptamer that binds only to either the Y chromosome-bearing mammalian sperm cells or the X chromosome-bearing mammalian sperm cells.

18. The method of claim 17, wherein said first and second samples of mammalian sperm cells are produced by flow cytometry and cell sorting.

19. The method of claim 17, wherein the aptamers are produced by the process wherein the contacting step (a) further comprises:
(a1) contacting a first collection of different nucleic acid molecules with a target molecule contained in a first sample of Y chromosome-bearing mammalian sperm cells under conditions favorable for binding between said nucleic acid molecules and said Y chromosome-bearing sperm cells to form at least one complex comprising at least one nucleic acid molecule bound to at least one Y chromosome-bearing sperm cell, wherein each of said nucleic acid molecules comprises at least one segment of randomized nucleotide sequences; and
(a2) contacting a second collection of different nucleic acid molecules with a target molecule contained in a second sample of X chromosome-bearing mammalian sperm cells under conditions favorable for binding between said nucleic acid molecules and said X chromosome-bearing sperm cells to form at least one complex comprising at least one nucleic acid molecules bound to at least one X chromosome-bearing sperm cell, wherein each of said nucleic acid molecules comprises at least one segment of randomized nucleotide sequences; and
wherein recovery step (c) thereby produces aptamers for Y chromosome-bearing mammalian sperm cells and aptamers for X chromosome-bearing mammalian sperm cells.

20. The method of claim 19, wherein aptamers that bind to Y chromosome-bearing mammalian sperm are recovered.

21. The method of claim 19, wherein aptamers that bind to X chromosome-bearing mammalian sperm are recovered.

22. The method of claim 17, wherein prior to step (a), at least 1 round of contact of said nucleic acid molecules with an unsorted cell population comprising both X-chromosome-bearing mammalian cells and Y-chromosome-bearing mammalian cells occurs.

23. The method of claim 17, wherein said separating step (b) further comprises the step of separating said sperm cells bound to the aptamer from the aptamer and said recovering step (c) further comprises recovering the separated sperm cells.

24. The method of claim 17, wherein the mammalian sperm cells are cattle sperm cells.

25. The method of claim 17, wherein the mammalian sperm cells are human sperm cells.

26. The method of claim 17, wherein at least one aptamer comprises two or more different aptamers.

27. The method of claim 17, wherein the aptamer is bound to a non-magnetic bead or a magnetic bead.

28. A method for producing an aptamer that permits separation of Y chromosome-bearing mammalian sperm cells from X chromosome-bearing mammalian sperm cells comprising the steps of:
(a) obtaining a first sample of Y chromosome-bearing mammalian sperm cells and a second sample of X chromosome-bearing mammalian sperm cells;
(b) producing a first group of aptamers that bind to Y chromosome-bearing sperm cells in said first sample and a second group of aptamers that bind to X chromosome-bearing mammalian sperm cells in said second sample; and
(c) comparing said first and second groups of aptamers to identify by process elimination at least one aptamer that binds only to either the Y chromosome-bearing mammalian sperm cells or the X chromosome-bearing mammalian sperm cells.

29. An aptamer produced by claim 17.

30. The sperm cell population produced by the method of claim 17, or a kit for artificial insemination of a mammal containing said sperm cell population comprising sperm cells bound to said aptamer.

31. The sperm cell population produced by the method of claim 17, or a kit for artificial insemination of a mammal containing said sperm cell population comprising sperm cells unbound to said aptamer.

32. The sperm cell population or said kit of claim 30, wherein said sperm cells are cattle sperm cells.

33. The sperm cell population or said kit of claim 30, wherein said sperm cells are human sperm cells.

34. The sperm cell population or said kit of claim 30, comprising sperm cells containing a Y chromosome or a X chromosome.

35. A method for artificial insemination of a mammal comprising administering to said mammal the sperm cell population comprising sperm cells bound to said aptamer or unbound to said aptamer produced by the method of claim 17.

36. A method of separating mammalian sperm cells, comprising the steps of contacting the mammalian sperm cells with at least one aptamer that binds only to either the Y chromosome-bearing mammalian sperm cells or the X chromosome-bearing mammalian sperm cells and separating the sperm cells into two or more populations based on the ability of the sperm cells to bind to either of the said aptamers.

37. The method of claim 36, further comprising the steps of separating said sperm cells bound to the aptamer from the aptamer and recovering the separated sperm cells.

38. The method of claim 36, wherein the mammalian sperm cells are cattle sperm cells or human sperm cells.

39. The method of claim 36, wherein at least one of the sperm cell populations comprises sperm cells containing a Y chromosome or containing a X chromosome.

40. The method of claim 36, wherein at least one of the sperm cell populations comprises sperm cells unbound to the aptamer.

41. The method of claim 36, wherein at least one of the sperm cell populations comprises sperm cells bound to the aptamer.

42. The method of claim 36, wherein the aptamer comprises two or more different aptamers.

43. The method of claim 36, wherein the aptamer is bound to a non-magnetic bead or a magnetic bed.


SEQUENCE LISTING (5 sequences)

SEQ ID NO: 1 - DNA, 80 bp, Unknown
  Annealing primer
ctcctctgactgtaaccacgnnnnnnnnnnnnnnnnnnnnnnnnnnnnnnnnnnnnnnnnggcttctggactacctatgc

SEQ ID NO: 2 - DNA, 20 bp, Unknown
  F primer
ctcctctgactgtaaccacg

SEQ ID NO: 3 - DNA, 20 bp, Unknown
  R primer
ggcttctggactacctatgc

SEQ ID NO: 4 - DNA, 20 bp, Unknown
  Alexa 647 primer
ctcctctgactgtaaccacg

SEQ ID NO: 5 - DNA, 20 bp, Unknown
  Reverse primer
ggcttctggactacctatgc